(19) United States
(12) Patent Application Publication
(10) Pub. No.: US 2017/0087379 A1
(43) Pub. Date: Mar. 30, 2017

(54) LIGHT-ACTIVATED ACNE TREATMENT

(71) Applicant: Filip Sedic, Stockholm (SE)

(72) Inventor: Filip Sedic, Stockholm (SE)

(21) Appl. No.: 15/232,605

(22) Filed: Aug. 9, 2016

(30) Foreign Application Priority Data
Sep. 28, 2015 (CN) 201510624612.2
Sep. 28, 2015 (CN) 201520754045.8

(51) Int. Cl. A61N 5/06; A61H 23/02

(52) U.S. Cl. CPC A61N 5/0616; A61H 23/02; A61N 2005/0652; A61N 2005/0663; A61N 2005/0644; A61N 2005/0626; A61N 2005/0666; A61H 2201/10; A61H 2201/0157

(57) ABSTRACT

A skin treatment device uses blue-light therapy to treat acne or other skin conditions. The device includes a positioning mechanism that allows a user to position the device on a target treatment area. Once the device is positioned, a safety triggering mechanism of the device activates the blue light therapy if it detects that the device is touching human skin, ensuring that sensitive areas (e.g., eyes) are not exposed to the blue-light therapy. An embodiment of the device includes a timing circuit to monitor the operating time of the device and an automatic shutdown circuit to prevent over-exposure of the blue-light therapy. Other embodiments of the device include a micro-vibration motor to massage a user's skin.

### CROSS REFERENCE TO RELATED APPLICATIONS

This application claims the benefit of Chinese Patent Application No. 201520754045.8 filed Sep. 28, 2015, and Chinese Patent Application No. 201510624612.2 filed Sep. 28, 2015, each of which is incorporated by reference in its entirety.

### BACKGROUND

Skin health and appearance is an important aspect of many beauty regimens. Common skin care routines focus on the prevention and treatment of acne. While many factors may contribute to the formation of acne, it is primarily driven by the growth of bacteria, e.g. propionibacteria. Clinical studies have shown several therapeutic advantages of blue-light therapy on acne caused by bacteria, such as rapidly diminished inflammation, minimization of the formation of acne, and improved regeneration of cells. Current skin treatment devices are not suitable for at-home use due to size, lack of safety measures, lack of targeted treatment mechanisms, or lack of safety mechanisms. An effective skin treatment device should be a small, portable, easy-to-use device that includes targeted treatment and safety mechanisms.

### SUMMARY

A skin treatment device which uses blue light-emitting diode photo dynamic therapy to treat acne. In one embodiment, the device includes a positioning mechanism that allows a user to position the device on a target treatment area. Once the device is positioned, a safety triggering mechanism of the device activates the blue-light therapy if it detects that the device is touching human skin, ensuring that sensitive areas (e.g. eyes) are not exposed to the blue-light therapy. An embodiment of the device includes a timing circuit to monitor the operating time of the device and an automatic shutdown circuit to prevent over-exposure of the blue-light therapy. Other embodiments of the device include a micro-vibration motor to massage a user's skin. The device is battery-powered.

### BRIEF DESCRIPTION OF THE DRAWINGS

FIG. 1A illustrates a perspective view of a first embodiment of a skin treatment device.

FIG. 1B illustrates a perspective view of a second embodiment of a skin treatment device.

FIG. 2 illustrates multiple perspective views of a skin treatment device, according to one embodiment.

FIG. 3 illustrates an exploded, perspective view of components within a skin treatment device, according to one embodiment.

FIG. 4A illustrates a configuration of light-emitting diodes within a skin treatment device, according to one embodiment.

FIG. 4B illustrates an additional configuration of light-emitting diodes within a skin treatment device, according to one embodiment.

FIG. 5A illustrates an exploded view of a sub-assembly with light-emitting diodes, according to one embodiment.

FIG. 5B illustrates various pathways of light emitted by the light-emitting diodes within a skin treatment device, according to one embodiment.

FIG. 6 illustrates a perspective view of a sub-assembly with light-emitting diodes within a skin treatment device, according to one embodiment.

FIG. 7 illustrates a cross-sectional view of a skin treatment device, according to one embodiment.

FIG. 8 illustrates a sequence of events for operation of a skin treatment device, according to one embodiment.

FIG. 9 illustrates a sequence of events during operation of a skin treatment device, according to one embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

The skin treatment device, hereinafter referred to as “acne pen,” delivers blue-light therapy to treat and heal acne on a user's skin. A user can target the device at a pinpoint location, activate the blue-light therapy, and treat a problem area for a set amount of treatment time. The user can repeat this process on multiple desired locations. The device is referred to as an acne pen, but it can also be used to treat any other type of skin condition for which blue light therapy may be effective. It can be used to treat skin conditions on any surface of the skin, including face, back, arms, etc.

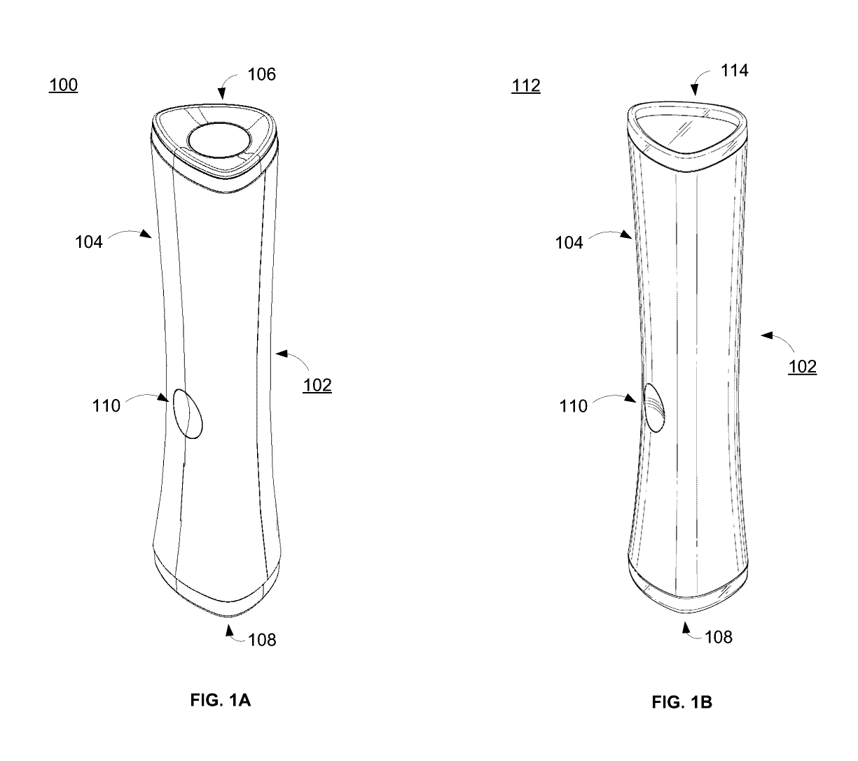

FIG. 1A illustrates a perspective view of a first embodiment of a skin treatment device. Some embodiments of the acne pen have different components than those described here. Similarly, in some cases, functions can be distributed among the components in a different manner than is described here. In the embodiment of FIG. 1A, the acne pen 100 comprises a housing 102, a housing cover 104, a top cover 106, a bottom cover 108, and a function button 110.

The housing 102 provides structural support for the acne pen 100. The housing 102 of the acne pen 100 can be configured to have different shapes, such as cylindrical, cubic, flashlight, pen, or any other form that conforms to the ergonomic features of a human hand. In the embodiment of FIG. 1A, the housing 102 has a cylindrical shape with a concave side wall, similar to a pen-type shape, which allows a user to easily hold the acne pen 100. The pen-type shape of the housing 102 is configured to be hand-held, ergonomic, and portable, which enables a user to accurately control the delivery of blue-light therapy. In the embodiment shown in FIG. 1A, the dimensions of the acne pen 100 are approximately 140 millimeters in length, 37 millimeters in width, and 32 millimeters in height. In other embodiments, the dimensions of the acne pen 100 may vary, e.g. fall within a range of 120-200 millimeters in length, 150-450 millimeters in width, and 100-450 millimeters in height, such that the acne pen 100 is configured to be hand-held, ergonomic, and portable. The housing 102 can be composed of various types of rigid materials (e.g. metal, glass, plastic, etc.). In the embodiment of FIG. 1A, the housing 102 is composed of plastic, which provides insulation for electricity, heat, and sound.

The housing cover 104 provides the user with a comfortable, non-slip grip. The housing cover 104 is tubular and is configured to form-fit to the shape of the housing 102, enclosing the full length of the housing 102. In the embodiment of FIG. 1A, the housing cover 104 is composed of a silica gel, which is non-absorbent, easy-to-clean, and durable. In other embodiments, the housing cover can be composed of various other elastic materials.

The top cover 106 is configured to be placed against a user's skin surface when the acne pen 100 is positioned to deliver blue-light therapy. The top cover 106 is secured to a top cover base at a first end of the housing 102 and is substantially triangular-shaped, such that the edges of the top cover 106 are substantially flush with the top cover base. In the embodiment of FIG. 1A, the top cover 106 is composed of a silicone material, which, in some embodiments, may have an antibacterial coating or have antibacterial properties to prevent the growth and spread of bacteria and/or other pathogens on the acne pen 100. As illustrated in the embodiment of FIG. 1A, a central part of the top cover 106 comprises a hole that is configured to allow the blue-light therapy to emit through the top cover 106 to a target treatment area.

The bottom cover 108 is configured to couple to a second end of the housing 102. The bottom cover 108 is substantially triangular-shaped, similar to the top cover 106, such that the edges of the bottom cover 108 are substantially flush with the outer circumference of the second end of the housing 102. The bottom cover 108 can be configured to act as a base to allow the acne pen 100 to stand upright. The bottom cover 108 can be composed of various types of rigid materials (e.g., metal, glass, plastic, etc.).

The function button 110 allows a user to control operation of the acne pen 100. The function button 110 may be a physical button, a button on a touch screen or a control panel, a sliding button, a knob, a switch, or the like. In the embodiment of FIG. 1A, the function button 110 is a button located along the length of the housing 102 and is operable through the housing cover 104. The function button 110 allows the user to control one or more functionalities of the acne pen 100, such as powering the acne pen on and off, increasing or decreasing the time of treatment, adjusting the intensity of the treatment, or switching between modes of operation. In some embodiments, the acne pen 100 can have various modes of operation, such as a massaging mode that can be used to massage the human body. The function button 110 can be configured to operate one mode, all modes, or some combination thereof. The function button 110 can be configured to allow a user to switch between two or more modes by pressing the function button 110 for specific durations of time (e.g., 1 second, 3 seconds, 5 seconds, etc.) or in a sequence of short presses (e.g., 1 press, 2 presses, 3 presses, etc.). The duration or sequence of presses may correspond to different modes of the acne pen 100.

FIG. 1B illustrates a perspective view of a second embodiment of a skin treatment device. Similarly, the functions and characteristics of the acne pen 100 can be incorporated for the acne pen 112. In the embodiment of 1B, the acne pen 112 has a varying structure for a top cover 114 that is different from the top cover 106 of FIG. 1A. The top cover 114 is secured to a top cover base at a first end of the housing 102 and is substantially triangular-shaped, such that the edges of the top cover 114 are substantially flush with the outer circumference of the first end of the housing 102. The top cover 114 can be composed of various types of rigid materials (e.g., metal, glass, plastic, etc.). In the embodiment of FIG. 1B, a central part of the top cover 114 is composed of a transparent material (e.g., glass, plastic, etc.). This configuration allows the blue-light therapy to emit through the top cover 114 to a target treatment area.

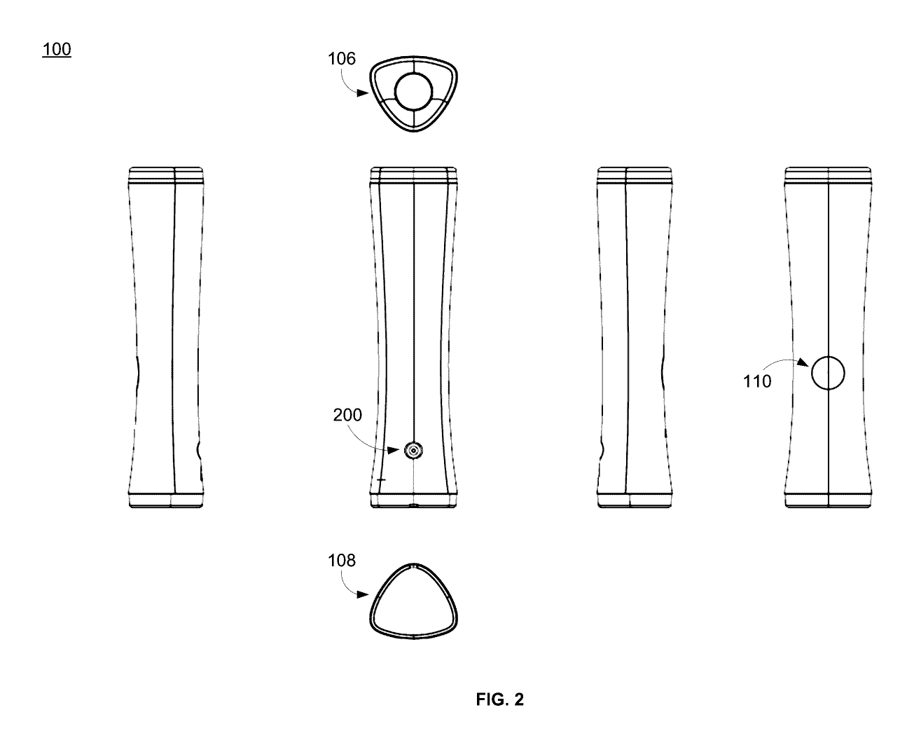

FIG. 2 illustrates multiple perspective views of a skin treatment device, according to one embodiment. As illustrated in the embodiment of FIG. 2, the acne pen 100 further comprises a charging port 200. The charging port 200 is configured to charge the power source within the acne pen 100. In the embodiment of FIG. 2, the charging port 200 is located along the length of the housing 102 on the opposite side from the function button 110. In other embodiments, the charging port 200 can be located on various sides of the housing 102 or on the bottom cover 108. FIG. 2 also illustrates the central hole or opening of the top cover 106 through which the blue-light therapy can emit, as previously described with regards to FIG. 1A.

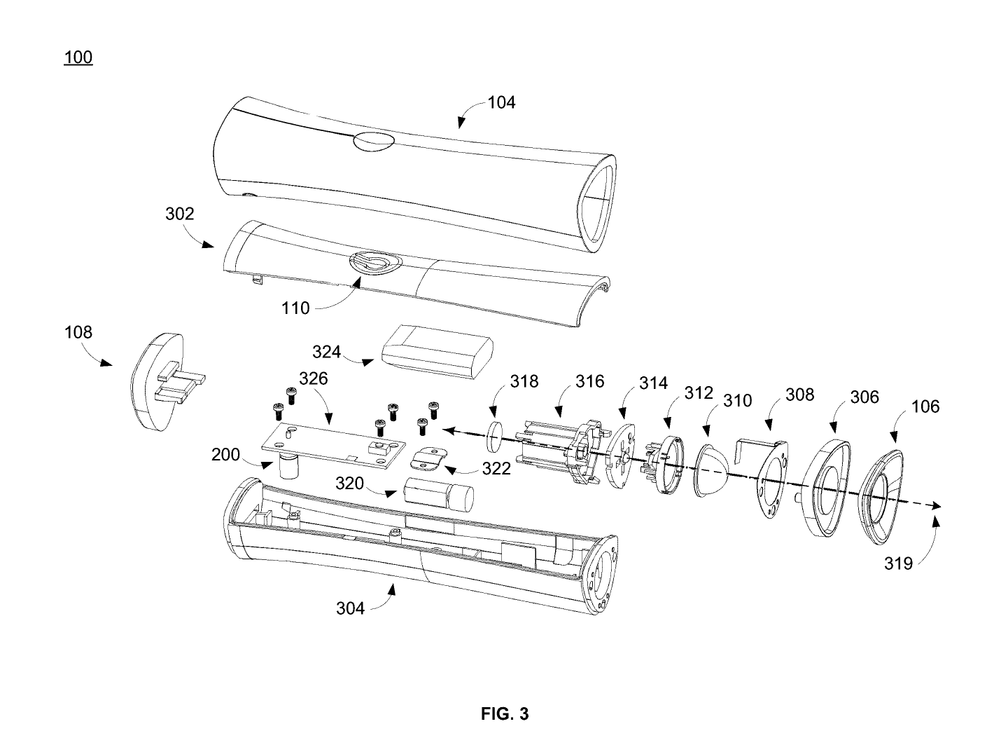

FIG. 3 illustrates an exploded view of components within a skin treatment device, according to one embodiment. Some embodiments of the acne pen 100 have different components than those described here. Similarly, in some cases, functions can be distributed among the components in a different manner than is described here. For example, the exploded view shown in FIG. 3 illustrates the components of the acne pen 100 as illustrated in the embodiment of FIG. 1A. The acne pen 100 has a varying structure for the top cover 106 that is different from the top cover 114 of FIG. 1B, as described with regards to FIGS. 1A and 1B. The acne pen 100 includes a plurality of external components and a plurality of internal components that will be described in further detail.

The external components of the acne pen 100 comprise the aforementioned housing 102, the housing cover 104, the top cover 106, the bottom cover 108, the function button 110, and the charging port 200. The housing 102 is comprised of an upper shell 302 and a lower shell 304. The upper shell 302 and the lower shell 304 couple to form the housing 102. In the embodiment of FIG. 3, the lower shell 304 has a cavity which secures the internal components of the acne pen 100. The upper shell 302 is configured to reciprocally secure to the lower shell 304.

The internal components of the acne pen 100 comprise a top cover base 306, a sensor 308, a lens 310, a lens holder 312, a light-emitting board (LEB) 314, an LEB holder, and a mirror 318, each aligned along an alignment axis 319. The internal components further comprise a vibration motor 320, a motor bracket 322, a battery 324, and a printed circuit board assembly 326.

The internal components form a positioning mechanism, a safety triggering mechanism, and a treatment mechanism. The positioning mechanism allows a user to accurately position the acne pen 100 at a treatment area to deliver blue-light therapy. The safety triggering mechanism ensures that blue-light therapy isn't delivered to sensitive areas, such as a user's eyes. The treatment mechanism delivers the blue-light therapy to a treatment area. Each of these mechanisms and the respective components involved will be discussed in further detail with regards to the following figures.

The vibration motor 320 is configured to create vibrations within the acne pen 100. In some embodiments, the acne pen 100 may have a micro-vibration massaging mode, in which a user uses the acne pen 100 to massage parts of the human body. The vibration motor 320 is mounted with a motor bracket 322 within the middle of the housing 102, such that the micro-vibrations of the vibration motor 320 are evenly distributed to the side walls of the acne pen, achieving optimal massaging effects. In other embodiments, the vibration motor 320 is located near an end of the housing 102, such that the micro-vibrations are focused at end to deliver targeted massaging effects. In some embodiments, the vibration motor 320 can be used to indicate the duration of treatment time to a user.

The battery 324 provides a power source for the acne pen 100. The battery 324 can have various forms, e.g. button cell, dry battery, or storage battery. In the embodiment of FIG. 3, the battery 324 is a lithium polymer battery, which is a type of storage battery, allowing the acne pen 100 to be used wirelessly. The battery 324 is secured within a cavity of the housing 102 via a battery holder and is configured to be charged via the charging port 200. In some embodiments, the acne pen 100 may powered by a wire electrical source or by a combination of the power sources described herein.

In some embodiments, the acne pen 100 may have one or more LEDs (not shown) located on the outside of the housing 102. The one or more LEDs act as an indicator and may indicate treatment time, treatment intensity, battery power levels, mode setting, or any combination thereof. The one or more indicator LEDs can be a variety of colors and sizes and can be arranged such that the LEDs illustrate a progression bar or level. The type of indication displayed by the one or more LEDs is in response to commands from the printed circuit board assembly 326.

The printed circuit board assembly (PCBA) 326 controls the operation of the acne pen 100. In the embodiment of FIG. 3, the PCBA 326 is configured to receive one or more requests from the user via the function button 110 and, in response to the one or more requests, the PCBA 326 sends commands to the appropriate internal components to execute the request. For example, the user may press the function button 110 to power on the acne pen 100, the request is relayed to the PCBA 326, and the PCBA 326 may command the battery 324 to power on the acne pen 100. In some embodiments, the PCBA 326 comprises a timing circuit and an automatic shutdown circuit.

The timing circuit is configured to monitor the duration of time that the acne pen 100 delivers blue-light therapy. The timing circuit can help to inform a user of the passage of time while the acne pen 100 is in operation, or, in some cases, to prevent a user from experiencing over-exposure to blue-light therapy, which could lead to skin damage. The timing circuit may send signals to the PCBA 326 to activate the one or more indicator LEDs (not shown) at certain time intervals to keep a user visually informed and reminded of the duration of treatment. In some embodiments, the timing circuit may send signals to the PCBA 326 to activate the vibration motor 320 to tactilely alert the user of the duration of treatment.

The automatic shutdown circuit is configured to shut down the device after the acne pen 100 has delivered blue-light therapy for a specific duration of time. The automatic shutdown circuit prevents a user from experiencing harmful side effects from over-exposure to blue-light therapy. The automatic shutdown circuit may shut down the acne pen 100 in response to a user failing to adhere to the recommended treatment times or a user overlooking the LEDs that indicate the treatment time. In the embodiment of FIG. 3, the maximum treatment time is 3 minutes. The maximum treatment time may vary in other embodiments, depending on a variety of factors, such as the intensity of the blue-light therapy, prescribed treatment times, etc.

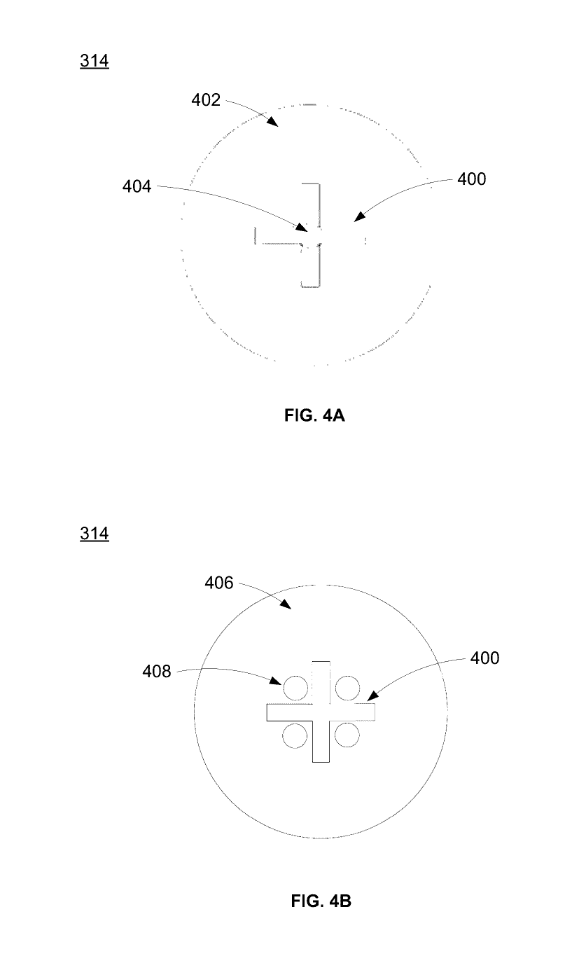

FIG. 4A illustrates a first face 402 of the LED light-emitting board (LEB) 314, according to one embodiment. The LED light-emitting board (LEB) 314 is configured to secure a plurality of light-emitting diodes (LEDs) within the acne pen 100. The LEB 314 is a circular disc composed of a rigid material (e.g. metal, plastic, etc.). In the embodiment of FIG. 4A, the LEB 314 comprises a cross-slot 400. The cross-slot 400 is a cross-shaped cut-out in the center of the LEB 314. On the first face 402 of the LEB 314, an LED 404 is coupled to the center of the cross-slot 400 via four points. In the embodiment of FIG. 4A, the LED 404 emits red light, which is used for the positioning mechanism to create a positioning mark. In other embodiments, the number, type, and configuration of LEDs 404 may vary. The LEDs 404 may be coupled to the LEB 314 through a variety of securing mechanisms, such as adhesive, solder, mechanical fasteners, or any other suitable securing mechanism.

FIG. 4B illustrates a second face 406 of the LED light-emitting board (LEB) 314, according to one embodiment. On the second face 406 of the LEB 314, a plurality of LEDs 408 is coupled to the LEB 314, such that one LED 408 is positioned within a quadrant constituted by the cross-slot 400. In the embodiment of FIG. 4B, the LEDs 408 emit blue light, which is used for the treatment mechanism to deliver blue-light therapy. In other embodiments, the number and configuration of LEDs 408 may vary. The LEDs 408 may be coupled to the LEB 314 through a variety of securing mechanisms, such as adhesive, solder, mechanical fasteners, or any other suitable securing mechanism.

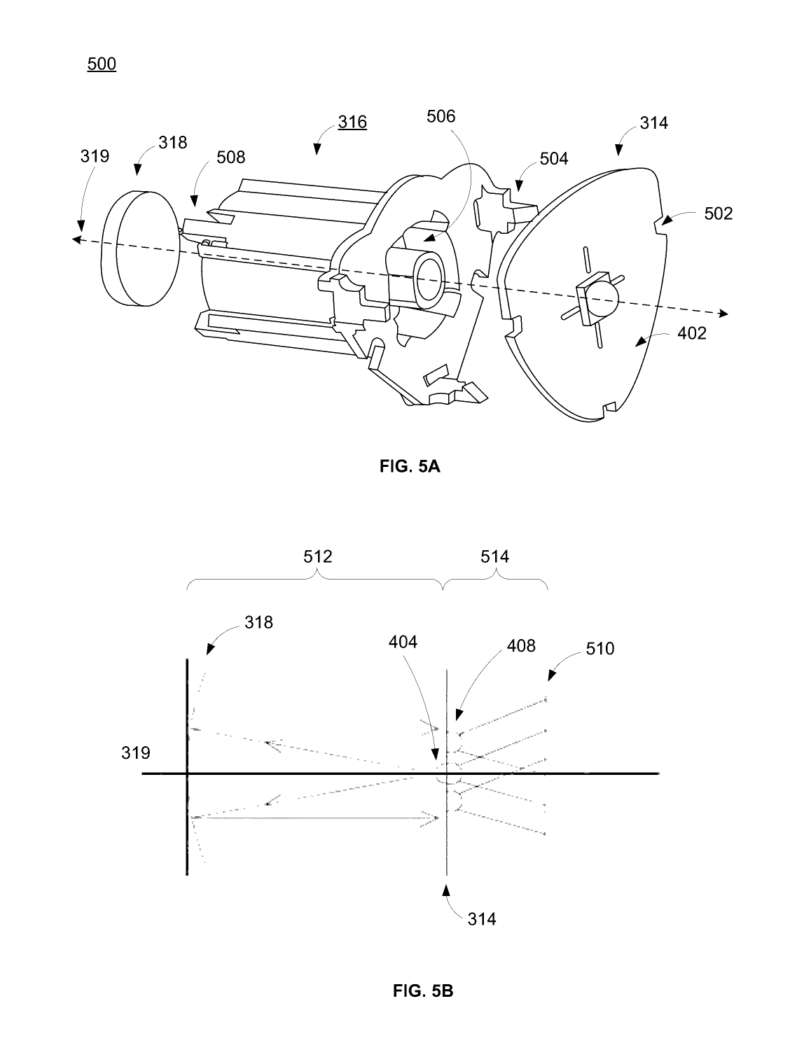

FIG. 5A illustrates an exploded view of a light-emitting board (LEB) assembly 500, according to one embodiment. The LEB assembly 500 is configured to maintain internal components in alignment along the alignment axis 319, such that the positioning mechanism and treatment mechanism can properly function. As previously mentioned with regards to FIGS. 4A and 4B, the positioning mechanism and the treatment mechanism both utilize the light emitted by the plurality of LEDs 404, 408 coupled to opposite faces of the LEB 314. In the embodiment of FIG. 5A, the LEB assembly 500 includes the LEB 314, the mirror 318, and the LEB holder 316.

The LEB 314 is configured to couple to a plurality of LEDs 404 and LEDs 408 within the acne pen 100, as previously described in the embodiments of FIGS. 4A and 4B. In the embodiment of FIG. 5A, the LEB 314 is substantially triangular-shaped with rounded corners. This embodiment illustrates a single LED secured to the center of the LEB 314, and the cross-slot 400 begins at the outer edges of the LED and extends towards the outer edge of the LEB 314. The LEB 314 includes a plurality of notches 502 located around the outer edge of the LEB 314. Each notch 502 is configured to reciprocally mate with securing tabs 504 on a first end of the LEB holder 316.

The mirror 318 is configured to reflect the light emitted by one or more LEDs coupled to the second face 406 (not shown in FIG. 5A) of the LEB 314, in the embodiment of FIG. 5A. The mirror 318 is substantially circular and is configured to reciprocally mate with securing tabs 508 on a second end of the LEB holder 316. The mirror 318 may be composed of a reflective material (e.g., glass, plastic, etc.). In the embodiment of FIG. 5A, the mirror 318 is concave and has an angle of incidence such that light reflects from the mirror 318 parallel to the alignment axis 319. In other embodiments, the mirror 318 may vary in concavity or convexity, given that the configuration of the mirror reflects light parallel to the alignment axis 319.

The LEB holder 316 is configured to secure the LEB 314 and the mirror 318 along the alignment axis 319. The LEB holder 316 includes a first end, a second end, and a tunnel 506. The first end of the LEB holder 316 has a substantially triangular face that includes a plurality of securing tabs 504, which reciprocally mate with the plurality of notches 502 of the LEB 314 and allow the LEB 314 to couple to the LEB holder 316. The second end of the LEB holder 316 has a substantially circular face that includes a plurality of securing tabs 508 to securely couple the mirror 318 to the LEB holder 316. The tunnel 506 extends through the center of the LEB holder 316 between the first end that secures the LEB 314 and the second end that secures the mirror 318. The tunnel 506 allows the light emitted by the LEDs secured to the face of the LEB 314 facing towards the LEB holder 316 to travel through the tunnel 506 towards the mirror 318, reflect off of the mirror 318, and travel through the tunnel towards the LEB 314. The configuration of the LEB holder 316 illustrated in FIG. 5A ensures that the LEB 314 and the mirror 318 are in alignment to avoid displacement between the two components during operation of the acne pen 100 and disrupt the functionality of the positioning mechanism or the treatment mechanism.

FIG. 5B illustrates the pathways of light emitted by the light-emitting diodes that contribute to the functionality of the positioning mechanism and the treatment mechanism, according to one embodiment. FIG. 5B illustrates the alignment of the LEB 314 with the mirror 318 that is provided by the LEB holder 316, as described in the embodiment of FIG. 5A. In the embodiment of FIG. 5B, the first face 402 of the LEB 314 includes a plurality of blue LEDs 408 and faces towards a target treatment area 510. The second face 406 of the LEB 314 includes a red LED 404 and faces towards the mirror 318. In some embodiments, the configuration of the LEB 314 may be reversed, such that the face including a plurality of red LEDs faces towards the target treatment area 510, and the face including a plurality of blue LEDs faces towards the mirror 318.

The positioning mechanism, as previously described with regards to FIG. 3, allows a user to accurately position the acne pen 100 at a target treatment area 510. In the embodiment of FIG. 5B, the LED 404 emits red light 512 through the tunnel 506 (not shown in FIG. 5B) in the direction of the mirror 318. The mirror 318 reflects the red light 512 parallel to the alignment axis 319 and back through the tunnel 506 (not shown in FIG. 5B). The red light 512 passes through the cross-slot 400 of the LEB 314 and emits through the top cover base 306 and the top cover 106 of the acne pen 100. In the embodiment of FIG. 5B, the red light 512 forms a cross-shaped positioning mark, which shines a cross-shaped positioning mark on the user's skin that the user uses to position the acne pen 100 at the targeted treatment area 510. For example, the user can adjust the acne pen 100 until the red cross-shaped positioning mark is positioned such that that acne portion to be treated is at the center of the cross when the user places the acne pen 100 against the user's skin. Thus, when the blue light is activated, it is treating the acne portion of the skin that the user wishes to treat. The LEB holder 316 maintains accurate alignment between the LEB 314 and the mirror 318 such that the red light 512 is able to form the cross-shaped positioning mark. In other embodiments, the LED 404 may be any color not harmful to the skin, eyes, or other sensitive areas of the human body (e.g., green, yellow, red).

The treatment mechanism, as previously described with regards to FIG. 3, delivers blue-light therapy to a target treatment area 510. The plurality of LEDs 408 emit blue light 514 through the top cover base 306 and the top cover 106 of the acne pen 100 to deliver blue-light therapy to the target treatment area 510. The light emitted by the LEDs 408 may be diffuse or targeted at a single point. Some embodiments may have a lens 310 and lens holder 312 positioned between the LEB assembly 500 and the sensor 308, as illustrated in the embodiment of FIG. 3, to intensify the blue-light therapy or to condense the blue-light therapy to a focal point.

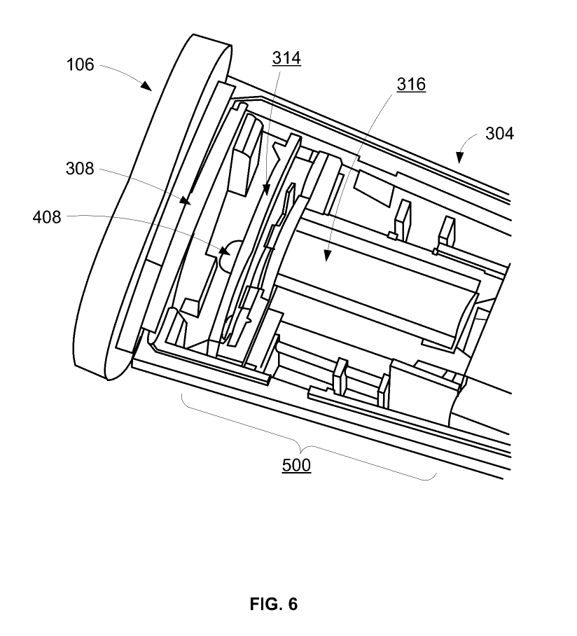

FIG. 6 illustrates a perspective view of the light-emitting board (LEB) assembly 500 within the acne pen 100, according to one embodiment. In the embodiment of FIG. 6, the LEB assembly 500 is shown secured within the lower shell 304 of the acne pen 100. This configuration of the LEB assembly 500 positions the LEB assembly 500 directly next to the sensor 308 and behind the top cover base 306 and the top cover 106 of the acne pen 100. A portion of the top cover base 306 is composed of a transparent material (e.g. glass, plastic, etc.) allowing light from the plurality of LEDs of the LEB 314 to emit through the top cover base 306 of the acne pen. The configuration of FIG. 6 further comprises a safety triggering mechanism.

The safety triggering mechanism allows a user to avoid exposing sensitive areas (e.g. a user's eyes) to the blue-light therapy. The sensor 308 is positioned between the LEB assembly 500 and the top cover base 306 and is coupled to the top cover base 306, which secures the top cover 106. In the embodiment of FIG. 6, the sensor 308 is a capacitor touch sensor, which is configured to detect objects that are conductive, such as a skin surface of a user. As previously described in some embodiments, the top cover 106 is configured to be placed against a user's skin surface when the acne pen 100 is positioned to deliver treatment. In the embodiment of FIG. 6, the sensor 308 is positioned at a certain distance from the top cover 106, such that the sensor 308 can detect when the top cover 106 has been placed against a user's skin surface. In other embodiments, the sensor 308 may be positioned with respect to the top cover base 306. In some embodiments, the distance between the sensor 308 and the top cover 106 may be between 2 mm to 5 mm. Upon detection of a skin surface, the sensor 308 is configured to send signals to the PCBA 326, which, in response, sends commands to de-activate the positioning mechanism and subsequently to activate the treatment mechanism. In this configuration, the blue-light therapy is delivered after the top cover 106 contacts a user's skin, allowing a user to avoid exposing sensitive areas to the blue-light therapy.

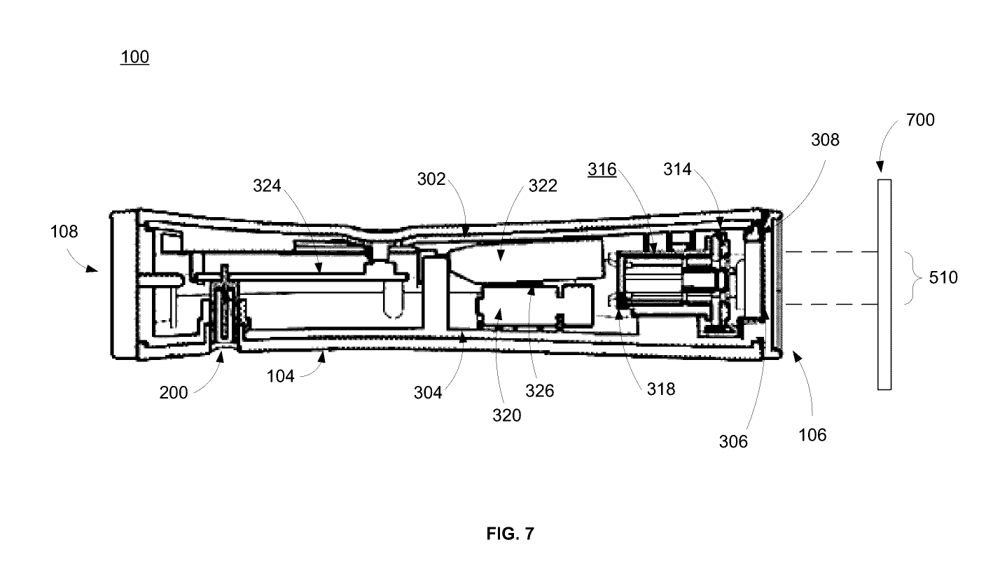

FIG. 7 illustrates a cross-sectional view of the acne pen 100, according to one embodiment. The external and internal components are shown assembled and in alignment. FIG. 7 also illustrates a user's skin surface 700 and a portion of the skin surface 700, which is a target treatment area 510. The target treatment area 510 aligns with the center portion of the top cover 106 from which the blue-light therapy can emit.

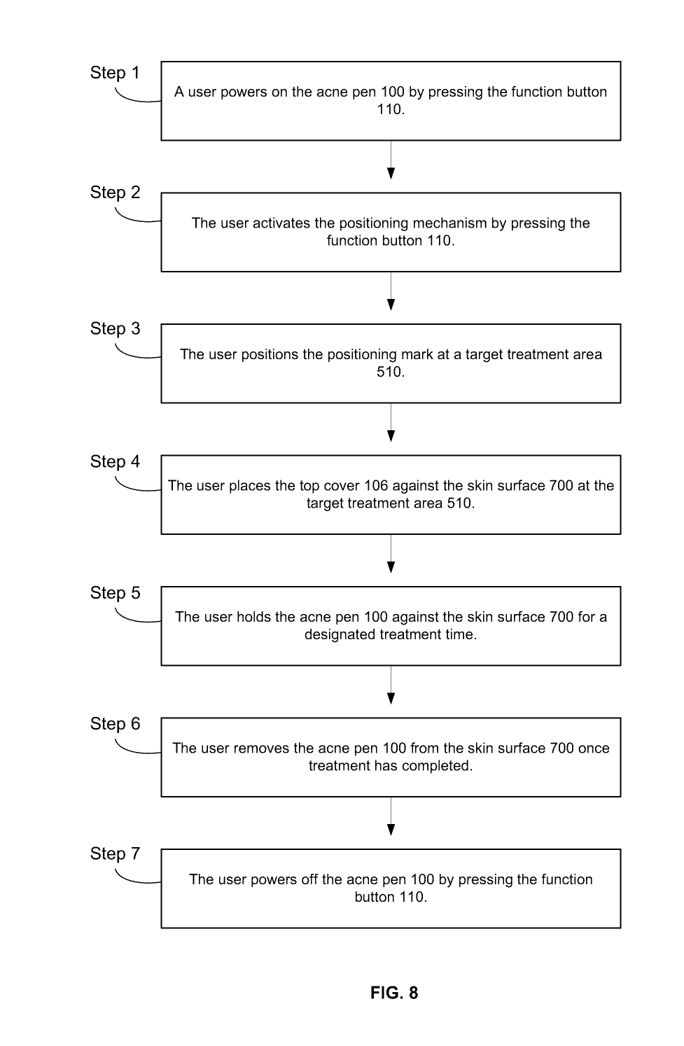

FIG. 8 illustrates a sequence of events for operation of a skin treatment device, including steps taken by a user of the device to operate the device, according to one embodiment. Some embodiments of the acne pen have different components than those described here. Similarly, in some cases, functions can be distributed among the components in a different manner and in a different sequence than is described here.

As described in Step 1, a user powers on the acne pen. In some embodiments, the user may activate the acne pen 100 by pressing the function button 110. A user may press the function button 110 a specific number of times or for a specified amount of time to activate the acne pen 100. In alternate embodiments, the user can activate the acne pen 100 by various other methods. Alternate methods may include placing the top cover 106 against a user's skin to activate the sensor 308 or by removing the acne pen from a charging dock.

As described in Step 2, the user activates the positioning mechanism after the acne pen 100 is powered on. In some embodiments, the positioning mechanism may be activated by the function button 110 or an additional button (e.g., button, switch, knob, or the like), such that the positioning mark appears when desired. A user may press the function button 110 a specific number of times or for a specified amount of time to activate the positioning mechanism. In alternate embodiments, the positioning mechanism may be activated immediately when the acne pen 10 is powered on. In the embodiment of FIG. 8, the positioning mechanism creates a cross-shaped positioning mark that allows a user to accurately position the acne pen 100 at a target treatment area 510. The positioning mechanism may be active if the acne pen 100 is powered on and the blue-light therapy is not being delivered.

As described in Step 3, the user positions the acne pen 100 in alignment with a target treatment area 510. In the embodiment of FIG. 8, the user uses the cross-shaped positioning mark created by the positioning mechanism to target a desired area for treatment.

As described in Step 4, the user places the top cover 106 of the acne pen 100 in contact with the skin surface 700 that surrounds the target treatment area 510. When in contact with a user's skin surface 700, the treatment mechanism of the acne pen 100 delivers blue-light therapy to a target treatment area 510. This ensures that blue-light therapy is not unintentionally delivered to sensitive areas (e.g. eyes, etc.).

As described in Step 5, the user holds the acne pen 100 against the skin surface 700 at the target treatment area 510 for a designated treatment time. In some embodiments, as the treatment time elapses, an indicator (e.g., an LED or a vibration motor) may activate each time a specific interval of time has passed to inform the user of the duration of treatment time. The user may deliver blue-light therapy to the target treatment area 510 for the maximum treatment time or less. In some embodiments, once the maximum treatment time has been reached, the treatment mechanism may automatically deactivate.

As described in Step 6, the user removes the acne pen 100 from contact with the user's skin surface 700 once treatment has completed. Removing the acne pen 100 from contact with the user's skin surface 700 deactivates the blue-light therapy, and the positioning mark reappears. In some embodiments, the positioning mechanism may not activate automatically and may require the user to activate it. The user may repeat Steps 3-6 for multiple target treatment areas 510 as desired.

As described in Step 7, the user powers off the acne pen 100 once treatment of all desired target treatment areas 510 is finished. In alternate embodiments, the user may choose to switch the mode of the acne pen 100 and continue to use the acne pen 100 in a different mode, such as a massaging mode. Once the user is finished using the acne pen 100, the user may press the function button 110 a specific number of times or for a specified amount of time to power off the acne pen 100. For some embodiments, the user can place the acne pen 100 onto a charging dock or plug a power source into the charging port 200 to recharge the battery 324.

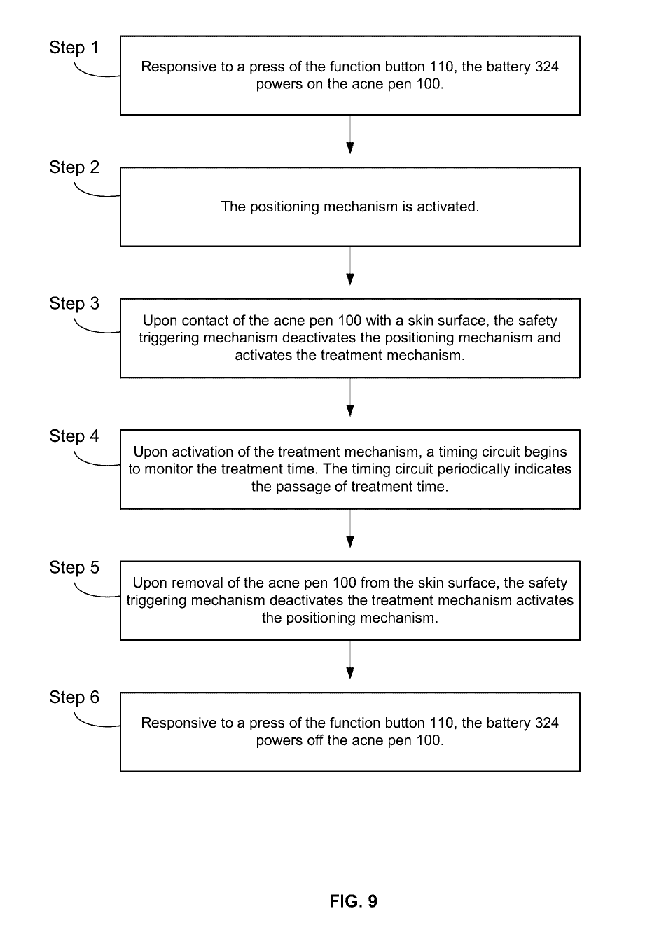

FIG. 9 illustrates a sequence of events during operation of a skin treatment device, including events that occur from the standpoint of the device, according to one embodiment. Some embodiments of the acne pen have different components than those described here. Similarly, in some cases, functions can be distributed among the components in a different manner and in a different sequence than is described here.

As described in Step 1, the acne pen 100 is powered on. In some embodiments, the battery 324 is activated in response to a user pressing the function button 110. In alternate embodiments, the battery 324 can be activated by various other methods. Alternate methods may include in response to activation of the sensor 308 or removal of the acne pen 100 from a charging dock.

As described in Step 2, the positioning mechanism is activated after the acne pen 100 is powered on. In some embodiments, the positioning mechanism may be activated in response to a user pressing the function button 110 or an additional button (e.g. button, switch, knob, or the like), such that the positioning mark appears when desired. A user may press the function button 110 a specific number of times or for a specified amount of time to activate the positioning mechanism. In alternate embodiments, the positioning mechanism may be activated immediately in response to the acne pen 10 being powered on. In the embodiment of FIG. 8, red light from the LEDs 404 is emitted, reflected off the mirror 318, and emitted through the cross-slot 400 of the LEB 314 and through the top cover 106. The positioning mechanism creates a cross-shaped positioning mark that allows a user to accurately position the acne pen 100 at a target treatment area 506. The positioning mechanism is active if the acne pen 100 is powered on and the blue-light therapy is not being delivered.

As described in Step 3, upon contact of the acne pen 100 with a user's skin surface, the safety triggering mechanism deactivates the positioning mechanism and activates the treatment mechanism (though in some embodiments, the positioning mechanism may remain on or may deactivate after a period of time). In the embodiment of FIG. 9, the safety triggering mechanism is triggered in response to the top cover 106 of the acne pen 100 contacting a user's skin surface and the sensor 308 detecting the conductivity of the user's skin surface. This ensures that blue-light therapy is not unintentionally delivered to sensitive areas (e.g. eyes, etc.). While the treatment mechanism of the acne pen 100 is activated, the blue-light therapy is delivered to a target treatment area 510.

As described in Step 4, upon activation of the treatment mechanism, a timing circuit begins to monitor the treatment time. The timing circuit monitors the duration of time that blue-light therapy is being delivered. In some embodiments, as the treatment time elapses, an indicator (e.g. an LED or a vibration motor) may activate each time a specific interval of time has passed. Some embodiments may have an automatic shutdown circuit, such that the treatment mechanism which delivers blue-light therapy will deactivate once the timing circuit detects that a maximum treatment time has been reached. The automatic shutdown circuit may help to prevent over-exposure to blue-light therapy.

As described in Step 5, upon removal of the acne pen 100 from a user's skin surface, the safety triggering mechanism deactivates the treatment mechanism and activates the positioning mechanism such that blue-light therapy is not delivered and the positioning mark reappears. This event occurs in response to the conductive circuit of the sensor 308 being broken when not in contact with a skin surface. In some embodiments, the positioning mechanism may not activate automatically after the treatment mechanism is deactivated and may require the user to activate it.

As described in Step 6, the battery 324 of the acne pen 100 is powered off in response to a user pressing the function button 110. This event occurs once treatment of all desired treatment areas is completed or use of the acne pen 100 in a different mode is completed. For some embodiments, the battery 324 may deactivate when the acne pen 100 is placed onto a charging dock or a power source is plugged into the charging port 200 to recharge the battery 324.

### SUMMARY

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

What is claimed is:

1. A skin treatment device comprising:
a housing configured to be hand-held, the housing comprising an upper casing configured to be attached to a lower casing, the housing further comprising a first end composed of a transparent material;
a positioning mechanism secured within the housing, the positioning mechanism comprising:
a plurality of colored light-emitting diodes, and
a reflective mirror configured to reflect light of the colored light-emitting diodes such that the light is emitted through the first end of the housing;
a plurality of blue light-emitting diodes secured within the housing, the plurality of blue light-emitting diodes arranged for emitting blue light through the first end of the housing;
a safety triggering mechanism comprising a sensor within the first end of the housing, the sensor configured to be activated by contact with a skin surface, the safety triggering mechanism configured such that, responsive to activation of the sensor, the plurality of colored light-emitting diodes power off and the plurality of blue light-emitting diodes power on successively; and
at least one control on the housing configured to operate a plurality of functions of the skin treatment device.

2. The skin treatment device of claim 1, wherein the sensor of the safety triggering mechanism is a capacitor touch sensor.

3. The skin treatment device of claim 1, wherein the plurality of colored light-emitting diodes and the plurality of blue light-emitting diodes are secured to opposite faces of a light-emitting diode board (LEB).

4. The skin treatment device of claim 3, wherein the LEB is coupled to the reflective mirror such that both are aligned along an alignment axis and that the reflective mirror reflects the light of the plurality of colored light-emitting diodes along the alignment axis.

5. The skin treatment device of claim 1, wherein the skin treatment device comprises a timing circuit within the housing to monitor the duration of time that blue light is emitted through the first end of the housing.

6. The skin treatment device of claim 5, wherein the timing circuit is configured to activate one or more indicators on the skin treatment device to alert a user of elapsed time intervals that blue light is emitted through the first end of the housing.

7. The skin treatment device of claim 5, wherein the skin treatment device comprises an automatic shutdown circuit, such that the blue light is deactivated once the timing circuit detects that the duration of time that blue light is emitted through the first end of the housing has passed a threshold time.

8. The skin treatment device of claim 1, wherein the skin treatment device comprises a vibration motor secured within the skin treatment device configured to deliver massaging effects to a user.

9. The skin treatment device of claim 1, wherein the plurality of colored light-emitting diodes of the positioning mechanism form a cross-shaped positioning mark.

10. The skin treatment device of claim 1, wherein the housing of the skin treatment device is encased within a silica gel cover.

11. A skin treatment device comprising:
a housing configured to be hand-held, the housing comprising a first end composed of a transparent material;
a plurality of blue light-emitting diodes secured within the housing, the plurality of blue light-emitting diodes arranged for emitting blue light through the first end of the housing;
a safety triggering mechanism comprising a sensor within the first end of the housing, the sensor configured to be activated by contact with a skin surface, the safety triggering mechanism configured such that, responsive to activation of the sensor, the plurality of blue light-emitting diodes power on; and
at least one control on the housing configured to operate a plurality of functions of the skin treatment device.

12. The skin treatment device of claim 11, wherein the sensor of the safety triggering mechanism is a capacitor touch sensor.

13. The skin treatment device of claim 11, wherein a positioning mechanism is secured within the housing, the positioning mechanism comprising:
a plurality of colored light-emitting diodes, and
a reflective mirror configured to reflect light of the colored light-emitting diodes such that the light is emitted through the first end of the housing;

14. The skin treatment device of claim 13, wherein the plurality of colored light-emitting diodes are configured to be powered off by the safety triggering mechanism in response to activation of the sensor.

15. The skin treatment device of claim 13, wherein the plurality of colored light-emitting diodes and the plurality of blue light-emitting diodes are secured to opposite faces of a light-emitting diode board (LEB).

16. The skin treatment device of claim 13, wherein the LEB is coupled to the reflective mirror such that both are aligned along an alignment axis and that the reflective mirror reflects the light of the plurality of colored light-emitting diodes along the alignment axis.

17. The skin treatment device of claim 11, wherein a timing circuit within the housing monitors the duration of time that blue light is emitted through the first end of the housing and is configured to activate one or more indicators on the skin treatment device to alert a user of elapsed time intervals that blue light is emitted through the first end of the housing.

18. The skin treatment device of claim 17, wherein the skin treatment device comprises an automatic shutdown circuit within the housing, such that the blue light-emitting diodes are powered off once the timing circuit detects that the duration of time that blue light is emitted through the first end of the housing has passed a threshold time.

19. A method for operating a skin treatment device comprising:
activating a positioning mechanism comprising a plurality of colored light-emitting diodes for allowing a user to position a treatment area of the skin treatment device on a skin surface of the user;
receiving an indication that a sensor configured to detect the skin surface of the user is activated based on the sensor having detected the skin surface on or near the treatment area of the skin treatment device;
responsive to activation of the sensor, deactivating the positioning mechanism and activating a plurality of blue-light emitting diodes for treatment of the skin surface;
responsive to deactivation of the sensor based on the sensor no longer detecting the skin surface on or near the treatment area of the skin treatment device, deactivating the plurality of blue-light emitting diodes.

20. The method of claim 19, further comprising monitoring the duration of time that the plurality of blue-light emitting diodes are active.

21. The method of claim 20, further comprising indicating to a user the duration of elapsed time that the plurality of blue-light emitting diodes are active.

22. The method of claim 20, further comprising deactivating the plurality of blue-light emitting diodes when the duration of elapsed time passes a threshold time.

23. The method of claim 19, further comprising activating the plurality of colored light-emitting diodes in response to deactivation of the sensor.
